(19) United States
(12) Patent Application Publication
(10) Pub. No.: US 2025/0198982 A1
(43) Pub. Date: Jun. 19, 2025

(54) SYSTEM AND METHOD FOR OPTOGENETICS STIMULATION OF NEURAL TISSUES ON MICROELECTRODE ARRAYS

(71) Applicant: The Regents of the University of California, Oakland, CA (US)

(72) Inventors: Kateryna Voitiuk, Santa Cruz, CA (US); Mircea Teodorescu, Santa Cruz, CA (US); Jessica Sevetson, Santa Cruz, CA (US)

(21) Appl. No.: 18/848,556

(22) PCT Filed: Apr. 4, 2023
(86) PCT No.: PCT/US2023/017491

§ 371 Date: Sep. 19, 2024

Related U.S. Application Data
(60) Provisional application No. 63/327,008, filed on Apr. 4, 2022.

(51) Int. Cl. G01N 33/483; G01N 21/63; G01N 33/68

(52) U.S. Cl. CPC G01N 33/4836; G01N 21/63; G01N 33/68; G01N 2201/0484; G01N 2201/062; G01N 2201/08; G01N 2201/12

(57) ABSTRACT

An optogenetic stimulation and electrophysiological recording system includes an electrophysiological device coupled to optically active neural tissue, where the electrophysiological device has a microelectrode array (MEA) configured to measure electrophysiological signals. The system also includes an optical stimulation device configured to emit light configured to stimulate the neural tissue. The system further includes a computing device coupled to the electrophysiological device and the optical stimulation device. The computing device is configured to control the optical stimulation device to emit the light to stimulate the neural tissue and simultaneously receive electrophysiological signals from the optically stimulated neural tissue through the electrophysiological device.

### CROSS-REFERENCE TO RELATED APPLICATION

The present application claims the benefit of and priority to U.S. Provisional Application No. 63/327,008, filed on Apr. 4, 2022. The entire contents of the foregoing application are incorporated by reference herein.

### GOVERNMENT LICENSE RIGHTS

This invention was made with government support under Grant No. R01MH120295, awarded by the National Institute of Mental Health of the National Institutes of Health, Grant No. 2034037, awarded by the National Science Foundation, and Grant No. RM1HG011543 awarded by the National Human Genome Research Institute of the National Institutes of Health. The Government has certain rights in the invention.

### BACKGROUND

Neurons are the building blocks of brains, and as such, the more we can understand their behavior and the connections they form with each other, the more we can understand the brain as a whole. Interfacing with neurons embedded in brain organoids that are derived from stem cells would allow for studying neurons in a contained environment.

In the realm of electrophysiology, a common technique for reading neural activity data is through a complementary metal-oxide semiconductor (CMOS) microelectrode array. These arrays consist of electrodes that can monitor electrical activity taking place on them; however older electrode arrays have a low density of total electrodes, making it harder to get more detailed and location specific activity data. More recently, the high-density (HD) microelectrode array (MEA) has become available and greatly increases the total amount of electrodes available in the same amount of space by decreasing their size, allowing for much higher resolution. As a tradeoff for this, most HD-MEAs cannot record data from all electrodes at once, however the benefits of the higher resolution electrodes can still be taken advantage of by pre-selecting which electrodes you want to monitor, since in most cases the culture does not cover the entire surface.

Organoids can be differentiated to mimic specific parts of the brain and behave as a miniature version of an actual brain area. Since organoids are three dimensional cultures, they mimic the functions of the brain and act as a closed system for monitoring and stimulation purposes. However, when paired with electrode-based sensors, the organoids' spherical shape makes it more difficult to monitor their electrophysiological activity due to the area of contact between the sensor and the organoid being smaller than that of a two-dimensional culture and a sensor. Even though this is the case, this drawback can help us gain an understanding of internal neural circuitry. For example, if an organoid is stimulated at the top and the sensor on the bottom is registering activity caused by this stimulation, it can be assumed that there is neural circuitry creating a consistent internal network.

Currently, there are no platforms that provide for simultaneous optogenetic stimulation and electrophysiological monitoring. In terms of optogenetics, bespoke light emitting diode (LED) panels are the most used design. While varying in scope, all these systems seek to stimulate neural cultures and then analyze and image them under a microscope. However, none of the existing designs combine the benefits of optogenetics with the form factor of the HD-MEA, which allows for high detail neural recording information and simultaneous stimulation and recording.

### SUMMARY

The present disclosure provides a platform for the optogenetic stimulation of cerebral organoids using optoelectrical equipment and an HD-MEA. The system includes a lighting assembly that may be used with a MEA. The system was validated through a series of experiments described in the “Examples” section using a fiber optic cannula to stimulate a human brain organoid infected with an adeno-associated virus (AAV) lentivirus to express channelrhodopsin-2 during neural recording sessions. Experimental procedures for stimulating and monitoring electrophysiological data from the organoids during neural activity recordings are also detailed in the “Examples.”

The disclosed optogenetic platform includes hardware and software and is configured to excite tissues on HD-MEAs. The platform may be used to characterize optogenetic stimulation on CMOS-based arrays, and provide solutions to common challenges (e.g., noise issues of nearby CMOS amplifiers, noise issues from sudden changes of light on CMOS array, etc.). The platform may be used to characterize neural response to optogenetic stimulation of different tissue types and explore the capability of different stimulation paradigms.

According to one embodiment of the present disclosure, an optogenetic stimulation and electrophysiological recording system is disclosed. The system includes an electrophysiological device coupled to optically active neural tissue, where the electrophysiological device has a microelectrode array (MEA) configured to measure electrophysiological signals. The system also includes an optical stimulation device configured to emit light configured to stimulate the neural tissue. The system further includes a computing device coupled to the electrophysiological device and the optical stimulation device. The computing device is configured to control the optical stimulation device to emit the light to stimulate the neural tissue and simultaneously record electrophysiological signals from the optically stimulated neural tissue through the electrophysiological device.

Implementations of the above embodiment may include one or more of the following features. According to one aspect of the above embodiment, the neural tissue may be an organoid expressing an opsin activatable by the light at a specific wavelength. The opsin may be one of halorhodopsin, archaerhodopsin, leptosphaeria rhodopsin, a channelrhodopsin, or derivatives thereof. The electrophysiological device may include a well with the MEA disposed within the well, and the neural tissue disposed on the MEA. The optical stimulation device may include a light emitting diode (LED) configured to emit the light, a fiber optic cable coupled to the LED, and a well insert assembly insertable into the well and configured to position a distal end of the fiber optic cable to emit the light in a spot over the neural tissue disposed on the MEA. The LED may be configured to emit light having a wavelength from about 385 nm to about 625 nm. The computing device may further include a display screen configured to display a graphical user interface (GUI). The computing device may be further configured to generate a grid corresponding to the MEA based on the received electrophysiological signals, the grid may include a plurality of footprint units. The grid may be displayed on the GUI. Each footprint unit of the plurality of footprint units may include a waveform identifier selected from a plurality of waveform identifiers. The computing device may be further configured to generate a functional connectivity map between a plurality of neural units of the neural tissue. The functional connectivity map may also be displayed on the GUI.

According to another embodiment of the present disclosure, a method for optogenetic stimulation and electrophysiological recording is disclosed. The method includes emitting light through an optical stimulation device to optically stimulate neural tissue disposed on a microelectrode array (MEA) of an electrophysiological device. The method further includes simultaneously receiving electrophysiological signals from the optically stimulated neural tissue through the electrophysiological device at a computing device, where the computing device is configured to control the optical stimulation device and the electrophysiological device.

Implementations of the above embodiment may include one or more of the following features. According to one aspect of the above embodiment, the method may also include spike sorting the electrophysiological signals. The method may further include generating a grid corresponding to the MEA based on the received electrophysiological signals. The grid may include a plurality of footprint units. The method may further include displaying the grid on a graphical user interface (GUI) that is output on a display of the computing device. The method may additionally include generating a functional connectivity map between a plurality of neural units of the neural tissue. The method may further include displaying the functional connectivity map on the GUI that is output on the display of the computing device.

### BRIEF DESCRIPTION OF DRAWINGS

Various embodiments of the present disclosure are described herein below with reference to the figures wherein:

FIG. 1 is a schematic diagram of an optogenetic stimulation system according to an embodiment of the present disclosure;

FIG. 2 is a cross-sectional view of an optogenetic well insert assembly according to an embodiment of the present disclosure;

FIG. 3 is a schematic diagram of a computer architecture of the optogenetic stimulation system according to an embodiment of the present disclosure;

FIG. 4 is a bar graph of a configurable stimulation template according to an embodiment of the present disclosure;

FIG. 5 is an image of an organoid disposed on a CMOS HD-MEA of the optogenetic stimulation system according to an embodiment of the present disclosure;

FIG. 6 is a flow chart of a method for obtaining and processing electrophysiological data according to an embodiment of the present disclosure;

FIG. 7 is a plot of a spike raster with optogenetic stimulation events obtained using the optogenetic stimulation system according to an embodiment of the present disclosure;

FIG. 8 is a diagram of neural units and corresponding spatial footprint of the organoid stimulated using the optogenetic stimulation system according to an embodiment of the present disclosure;

FIG. 9 shows a plot of spike waveform of a stimulation signal overlayed across optogenetic stimulation events;

FIG. 10 shows a bar graph of distribution count of spike events of the stimulation signal overlayed across optogenetic stimulation events;

FIG. 11 shows a frequency of the stimulation signal overlayed across optogenetic stimulation events; and

FIG. 12 shows an interspike interval (ISI) distribution of spike times for a neuron stimulated using the optogenetic stimulation system according to an embodiment of the present disclosure.

### DETAILED DESCRIPTION

The present disclosure provides an optogenetics platform for stimulating cortical organoids while monitoring their response using a CMOS-based high-density microelectrode recording system that integrates high spatial and temporal resolution of the neural activity. The optogenetic platform allows neurons expressing opsins to receive light stimulation protocols, which are logged along with the neural activity data. The platform can be used to facilitate “closed loop” experiments, where optical stimulation can be administered based on neural activity monitored in real time. The platform incorporates 3D printed components for ease of reproducibility. The modularity of the system allows for selection of LEDs in the range of 385 nm-625 nm for different optogenetic actuators. In a specific example, an optical fiber coupled to a blue 475 nm LED may be used to stimulate organoids expressing channelrhodopsin-2 via pAAV-Syn-ChR2(H134R), which targets all neurons. Responses may be measured to different programmed stimulation protocols, which includes varying pulse frequencies, timings, and amplitudes. With cell-type specific channelrhodopsin expression, this platform provides the capability to perform closed loop experiments to understand the effects of neuron subtypes on the network and how perturbations affect responses in human neural circuits.

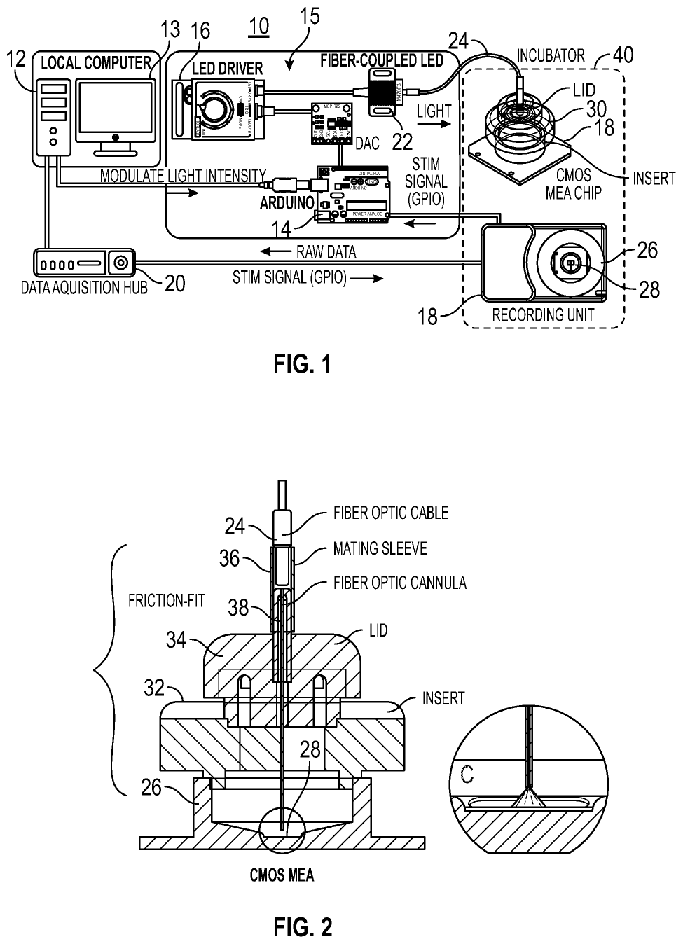

An optogenetic stimulation system 10 for stimulating and observing neurons disposed on an HD-MEA and observing neuron response to optogenetic stimulation is shown in FIG. 1. System 10 includes a computing device 12, which may be any suitable computing device such as, a desktop computer, a laptop, single-board computers (SBC), etc. The SBC may be Raspberry Pi, which provides a low cost, miniature computing platform. Computing device 12 may include a communication interface (e.g., ethernet, WiFi, etc.) allowing for communication with a network. Computing device 12 also includes a processor, a memory, a storage device, an input device, and a display screen 13. The processor is connected to each of the hardware components constituting the computing device 12.

The input device may be any suitable user input device such as a keyboard, a touch screen, a pointing device that can be operated by the operator and sends input signals according to an operation to the processor. The processor may be configured to perform operations, calculations, and/or sets of instructions described in the disclosure and may be a hardware processor, a field programmable gate array (FPGA), a digital signal processor (DSP), a central processing unit (CPU), a microprocessor, and combinations thereof. If an instruction is input by an operator using the input device, the processor executes a program stored in the memory. The processor is configured to load software instructions stored in the storage device and/or transferred from the network or a removable storage device (not shown) into the memory to execute such instructions. The memory may be a transitory storage device such as RAM (random access memory) and the like and is used as working memory for the processor and used to temporarily store data.

The storage device is a non-transitory storage device, e.g., hard disc drive, flash storage, etc. The storage device stores programs including application programs and an OS (operating system), as well as other data. The OS provides a GUI (graphical user interface) that displays information to the operator so that the operator can perform operations through the input device. The screen 13 may be any suitable monitor and may include a touchscreen that is configured to display the GUI for controlling the optogenetic stimulation system 10.

The computing device 12 is coupled to an interface device 14, which enables communication between the computing device 12 and optogenetic and electrophysiology stimulating components of the system 10, namely, an optical stimulation device 15 and an electrophysiological device 18, respectively.

The electrophysiological device 18 may be a MaxOne Single-Well MEA available from MaxWell Biosystems. The electrophysiological device 18 includes a well 26 (FIG. 2) and an MEA 28 disposed at the bottom of the well 26. The MEA 28 may be a suitable CMOS MEA having a plurality of electrodes, e.g., 26,400 (9.3×5.45 sq-μm, 17.5 μm pitch) and may have about 1,204 readout channels with 32 simultaneous channels. The electrophysiological device 18 is coupled to a data acquisition hub 20, which is configured to output a stimulation signal for the electrophysiological device 18, e.g., sampling rate amplitude, etc. The data acquisition hub 20 also measures neurological signals of the neural tissue disposed in the electrophysiological device 18 in response to electrical stimulating signals. The data acquisition hub 20 is coupled to the computing device 12, allowing the computing device 12 to receive, record, and process the neurological signals.

The optical stimulation device 15 includes an LED driver 16, which is also coupled to the interface device 14 via a digital-to-analog converter and is configured to output a drive signal for activating an LED 22, which may include one or more LEDs configured to output light at any desired wavelength or combination of wavelengths, which may be from about 385 nm to about 625 nm. The LED 22 is coupled via a fiberoptic cable 24 to an optogenetic well insert assembly 30, which is configured to be inserted into a well 26 of the electrophysiological device 18.

Electrophysiology systems, such as the electrophysiological device 18, may have general-purpose input/output (GPIO) pins or digital or analog input/outputs to send and transmit signals to external hardware components, such as TTL drivers for LEDs, sound systems for audio feedback, external user switches, buttons, etc. Thus, the output from the electrophysiological device 18 is passed through the interface device 14 and is used to drive the LED 22 for optogenetic excitation, i.e., through the LED driver 16.

With reference to FIGS. 1 and 2, an organoid is placed into well 26 and onto the MEA 28. FIG. 5 also shows the organoid (pointed via an arrow) placed on the MEA 28. The well insert assembly 30 is then inserted into the well 26. The well insert assembly 30 may include one or more portions, such as, an insert 32 configured to be inserted into the well 26 and a lid 34, insertable into the insert 32. The fiber optic cable 24 is terminated in a mating sleeve 36, which is inserted over a fiber optic cannula 38, which itself is inserted into the lid 34. Each of the components of the well insert assembly 30 (i.e., the insert 32, the lid 34, the mating sleeve 36, and the cannula 38) may be friction fit to allow for ease of assembly and disassembly of the well insert assembly 30. Each of the components of the well insert assembly 30 also include a centrally disposed opening therethrough to allow for assembly and alignment of the components, such that the light emitted by the fiber optic cable 24 forms a spot on the organoid placed over the MEA 28 as shown in an enlarged portion of FIG. 2.

The components of well insert assembly 30 may be formed using any additive techniques, such as 3D printing using MK3S Prusa 3D printer (PRUSA) or any other suitable 3D printer. Polylactic acid (PLA) such as Prusa Slic3r (PRUSA) or any other suitable polymers may be used. In embodiments, other 3D printable materials may be used, such as metals. The parts may be created with computer aided design (CAD) using any suitable application, such as Fusion 360 and AutoCAD (Autodesk). The components may be printed using infill settings from about 80% to about 100% with resolution of about 0.15 mm or higher.

The electrophysiological device 18 along with the well insert assembly 30 may be disposed inside an incubator 40 (FIG. 1), which may be any suitable cell culture incubator capable of maintaining preset humidity and temperature, e.g., humidity from about 75% to about 90% and temperature from about 35° C. to about 40° C.

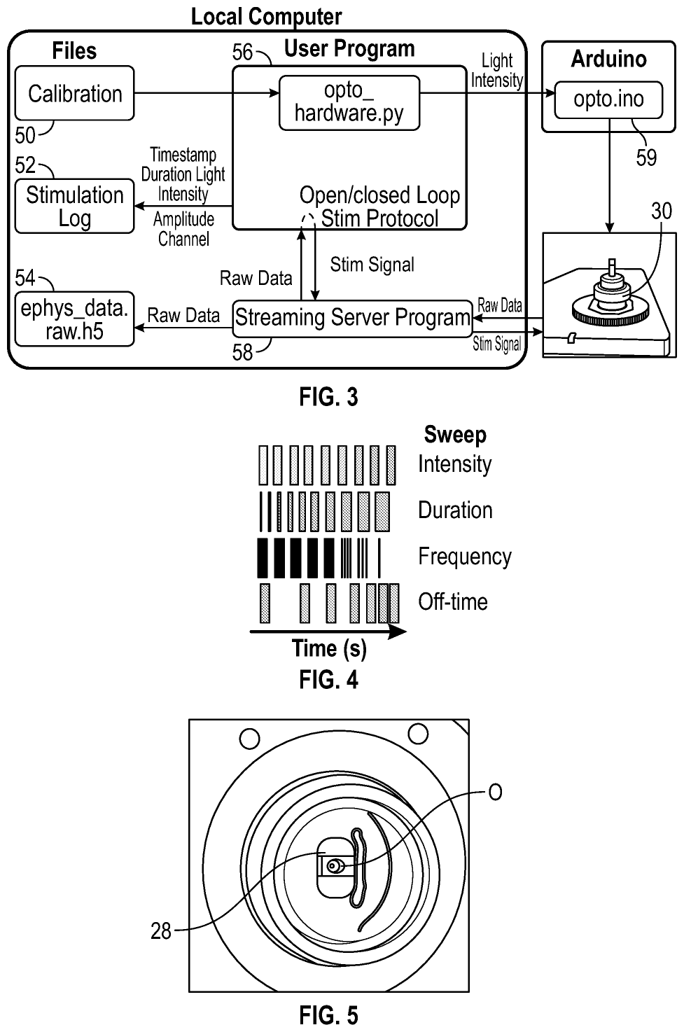

With reference to FIG. 3, a software architecture of system 10, including software modules executed by computing device 12, is shown. The computing device 12 stores a calibration file 50, which includes calibration parameters for LED power outputs; an optical stimulation log file 52 for storing data pertaining to optical stimulation signals delivered to the sample (i.e., timestamp, duration, light intensity, amplitude, channel, etc.); and an electrophysiological stimulation log file 54 storing electrophysiological stimulation signals delivered to the biological sample. Computing device 12 also stores a user program 56, e.g., one or more Python libraries, including software instructions and data flow supporting optogenetic and electrical stimulation control algorithms. The user program 56 communicates with a streaming server program 58 controlling the MEA 28 to output electrophysiological stimulation signals and to receive data signals. The user program 56 also communicates with software 59 of the interface device 14 controlling the output of the LED driver 16. The user program 56 also allows the user to construct their own stimulation sequences and take advantage of several helper functions with configurable stimulation pattern experiments such as those shown in FIG. 4 (e.g., varying intensity pattern, varying duration pattern, varying frequency pattern, varying off-time pattern, etc.).

The system 10 is used to monitor optogenetic response of an organoid including neurons expressing opsins. Suitable opsins include halorhodopsins (e.g., Jaws, Halo/NpHR, eNpHR 3.0, etc.), archaerhodopsins (e.g., Arch, eArch 3.0, ArchT, eArchT 3.0, etc.), leptosphaeria rhodopsins (e.g., Mac, eMac 3.0, etc.), channelrhodopsins (e.g., ChR2, ChR2/H134, ChETA, ChR/T159C, SFO/SSFO, ReaChR, VChR1, Chronos, Chrimson, ChrimsonR, PsChR2, CoChR, CsChR, CheRiff, C1C2, ChIEF, ChEF, ChD, C1V1, iChloC, SwiChRca, GtACR, PsChR1, Phobos, Aurora, etc.), or derivatives thereof. Organoids may be modified to express opsins using a viral vector (e.g., lentivirus) as is known to a person having ordinary skill in the art. In embodiments, the emitted light may be continuous (e.g., 1 minute or more), rather than short pulses, depending on the type of opsin being expressed.

The modified organoid is stimulated by the light provided by the LED 22 at a specific wavelength of the selected opsin(s), which may be from 385 nm to 625 nm. Once the organoid is stimulated, the electrophysiological activity is recorded by the MEA 28 and provided to the computing device 12, which is used to process and correlate the stimulation and electrophysiological signals.

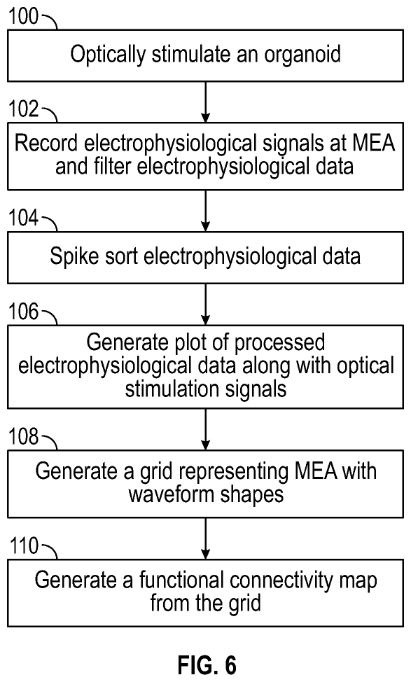

With reference to FIG. 6, a method for operating the system 10 includes applying light at a specific wavelength (e.g., 475 nm) to stimulate an organoid at step 100. Any suitable stimulation protocol described above may be used, e.g., intensity, duration, frequency, off-time patterns of FIG. 4. The method may be implemented as software instructions executable by the processor of the computing device 12.

At step 102, the electrophysiological signals in response to optical stimulation are recorded and are correlated to neuronal response by filtering the electrophysiological data with 300-6,000 Hz bandpass filter. At step 104, the data is spike sorted into single unit activity, which may be performed using Kilosort (see Pachitariu, M., Sridhar, S., & Stringer, C. (2023) “Solving the spike sorting problem with Kilosort,” bioRxiv, 2023-01). At step 104, the spike sorted data is further curated, e.g., accept, remove, split, or merge units based on their features like waveform template and interspike interval (ISI) distribution. In addition, noise-like units are also removed from the results to avoid false positive analysis.

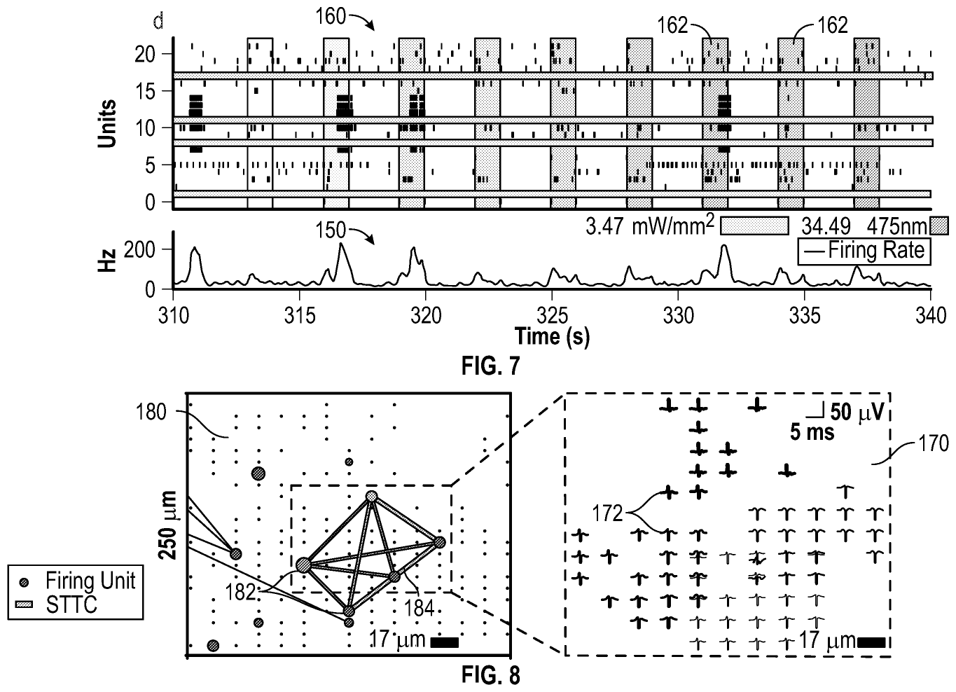

At step 106, the processed data is plotted to visualize the electrophysiological response along with the optical stimulation signals. With reference to FIG. 7, a spike raster plot 150 is shown along with a uniformly ascending intensity stimulation bar graph 160. The raster plot 150 shows electrophysiological response for all the firing units with their aggregated firing rate. The bar graph 160 includes a plurality of bars 162 indicating duration and intensity of the optical stimulation signals with increased saturation of the bars 162 denoting increased intensity. Optogenetic stimulation shown in bar graph 160 is aligned on the plot using bars 162 with a level of transparency (i.e., saturation) indicating the light intensity.

The MEA 28 includes multiple electrodes which pick up a signal from the same firing unit because of the narrow spacing between electrodes. Each recorded signal includes a waveform shape identifier, which may be color coded, and are plotted as footprint units 172 in a grid 170. Grid 170 represents the electrode array of the MEA 28 (FIG. 8) at step 108. More than one different waveform may occur at the same footprint unit 172. The waveform identifier on the footprint units 172 indicates that the selected units are from the same spiking neurons.

At step 110, grid 170 is then used to generate a functional connectivity map 180 including a plurality of neural units 182 (FIG. 8). Each neural unit 182 is defined from a plurality of the footprint units 172 from the grid 170. To find the correlation between footprint units 172, the spike time tiling coefficient (STTC) may be computed between all pairs of the footprint units 172 within a time window, e.g., 20 ms. The STTC may be from 0 to 1, where a more significant number means a higher correlation. In embodiments, the STTC threshold may be set to 0.35 to select the footprint units 172 with STTC passing this threshold and label them as functionally connected. The map 180 shows neural units 182 from a functionally connected network as well as lines 184 which show correlated activity between certain neural units 182. The opacity or saturation of the lines 184 is used to show the degree of correlation, i.e., the higher saturation denotes higher correlation. The grid 170, the connectivity map 180, and other items of steps 106-110 may be shown on the GUI displayed by the computing device 12.

The following Examples illustrate embodiments of the present disclosure. These Examples are intended to be illustrative only and are not intended to limit the scope of the present disclosure.

### Example 1

This Example describes organoid dissociation and 2D cell plating.

About 5-10 organoids (day 99) were dissociated using papain for 30 minutes on a shaker in an incubator (37° C., 5% CO₂). Organoids were transferred to a 15 mL conical, where papain was aspirated and replaced with CEPT (chroman 1, emricasan, polyamines, trans-ISRIB). This was followed by 15-20 triturations using glass fire-polished pipettes, after which organoids were spun at 150×G for 5 minutes, resuspended in 1 mL with CEPT, and counted. Meanwhile, MaxOne Single-Well MEA (MaxWell Biosystems) were incubated for 6 hours with Terg-a-zyme, rinsed, incubated for 30 min with 70% EtOH, rinsed, and incubated with 70 uL of 1% Matrigel for 1 hour, such that the electrode array was covered but Matrigel did not spill up onto the sides. Matrigel was aspirated off. Cells were resuspended in media with CEPT, plated on the MEA chips at a density of 300,000 cells in 75 uL, and incubated for 1 hr at 37° C., 5% CO₂ in a single-well plate with a 1 mL reservoir of autoclaved deionized H₂O for humidity. Following incubation, 500 uL media with CEPT was added. Media (without CEPT) was changed 2 times a week.

### Example 2

This Example describes organoid plating.

MaxOne MEA chips were incubated for 6-24 hours at about 25° C. with 1% Terg-a-zyme, rinsed, incubated for 30 min at RT with 70% EtOH, and rinsed with DMEM/F12. When ready to seed, 25 uL 1% v/v Matrigel in DMEM/F12 was added to each MEA chip. Using a P20 cut ⅔ of the way up, the organoids were transferred from the 6-well plate to the MEA well in 5 uL media. The media/Matrigel mixture was aspirated off until a minimal amount remained, and the chip was incubated at 37° C. for 1 hour in a 1-well plate with a reservoir of autoclaved deionized H₂O for humidity. Following incubation, 700 uL media was added. Media was changed by half volume, 2×/week. Each MEA well was covered with a sealing lid featuring a gas-permeable, water-impermeable membrane.

### Example 3

This Example describes generation of human brain organoids from human iPSCs.

Human H9 iPSCs were thawed into 6 cm plates coated with vitronectin and fed 5 mL StemFlex Medium with supplements every other day. The cell culture was expanded 3 times. Before aggregation, stem cells were passaged into a 10 cm plate and grown to 70% confluency. An Aggrewell800 plate was prepared and 1 mL of DMEM/F12 media was added to the plate. The plate was spun at 300 relative centrifugal field (rcf) for 2 min, media, was then aspirated and media wash was repeated for a total of 2 washes. Media was then aspirated and 1 mL of anti-adherence solution was added to each well. The plate was spun again at 300 rcf for 5 mins. Media was checked for bubbles and the plate was incubated at about 25° C. with anti-adherence solution for 45 minutes. After incubation, the anti-adherence solution was aspirated, 1 mL of Aggrewell media was added supplemented with CEPT, and the plate was spun at 300 rcf for 5 mins. The cell culture was dissociated, and 70% confluent stem cells were washed with 6 mL DPBS and aspirated. The cells were incubated with 3 mL accutase at 37° C. for 10 mins. Single cells resuspended in 3 mL Aggrewell+CEPT. Tubes had a combination of accutase, Aggrewell, and dmem/f12. Cell suspension was transferred to 15 mL tubes; each cell line had its own tube. The tubes were spun at 300 rcf for 3 mins. Supernatant was aspirated and resuspend in 6 mL Aggrewell+CEPT. Cell suspension was the counted and 8 uL of trypan blue and 8 uL of cell suspension were added to a 1.5 mL eppendorf tube. 8 uL of dye-cell solution was loaded into both sides of a Countess cell counter slide to confirm cell concentration 3×10\hat{ }6 cells/mL. Appropriate cell suspension volume was added to wells and brought up the volume to 2 mL of Aggrewell+CEPT Aggregate. The plate was spun at 100 rcf for 5 mins and incubated at 37° C. overnight. The plate was fed (i.e., 1 mL Aggrewell+sb/iwr1) the following day, then fed every day for 7 days.

### Example 4

This Example describes organoid plating and maintenance on MaxWell MEA chip.

Chips were cleansed with 1% Terg-a-zyme solution overnight (i.e., 15 mL tube, 8 mL milliQ, and 0.08 g Terg-a-zyme), shaken and stored submerged in PBS at 4° C. MEA chips were rinsed with media three times until the detergent was washed off. Electrodes were dried with an aspirator while aiming for the corner of the array to avoid touching the sensor. 5 uL of 6-10% Matrigel was added to the center of the electrodes. An organoid was transferred with a cut p20 tip and placed on the center of electrode array. Excess media was aspirated with a p200 and an extra 5 uL Matrigel was added on top of the organoid. The chip was incubated for 1 hour at 37° C. without media. After incubation test adherence, some media was slowly dropped into the well and it was noted whether perturbations move the organoid off the array. Once confirmed the organoid was successfully adhered, the well was slowly filled with 600 uL of media, incubated overnight, and covered with a sealing lid, featuring a gas-permeable, water-impermeable membrane.

### Example 5

This Example describes AAV8 virus infection.

The pAAV-syn-ChR2-GFP AAV8 virus (100 uL, titer: 3.3×10\hat{ }13) was diluted 1:500 for a higher working solution. 2 mL of media was added to a 15 mL tube and 4 uL of virus, and mixed. In the MaxWell chip, 300 uL of media was added to the chip, and brought up to 600 uL total volume by adding 300 uL of diluted AAV8 solution. The chips were stored in an incubator at 37° C. and fed 300 uL of media without virus twice a week.

### Example 6

This Example describes data analysis.

### Data Acquisition

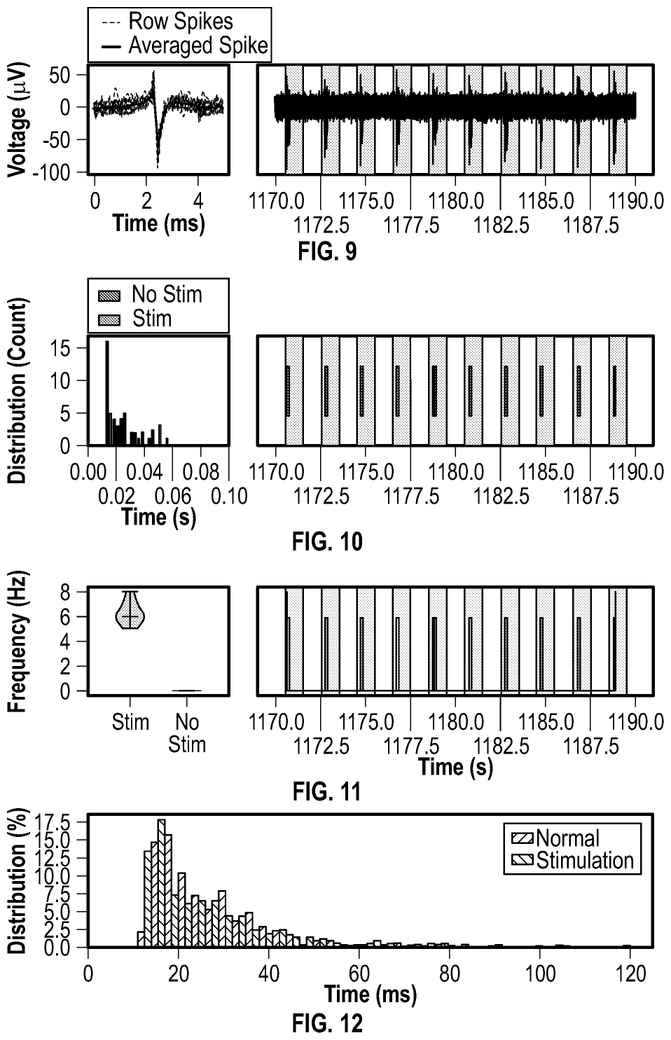

The optical fiber coupled to a blue 475 nm LED was used to stimulate organoids expressing channelrhodopsin-2 via pAAV-Syn-ChR2(H134R), which targets all neurons. Responses were measured to different programmed stimulation protocols, which included varying pulse frequencies, timings, and amplitudes. Neuronal firing data was recorded on the MaxWell MaxOne headstage with different light stimulation protocols. (See FIG. 4 and corresponding description above). A constant intensity stimulation protocol was used to obtain recordings shown in FIGS. 9-12, which show a plot of spike waveform of a stimulation signal overlayed across optogenetic stimulation events (FIG. 9); a bar graph of distribution count of spike events of the stimulation signal overlayed across optogenetic stimulation events (FIG. 10); a frequency of the stimulation signal overlayed across optogenetic stimulation events (FIG. 11); and an interspike interval (ISI) distribution of spike times for a neuron stimulated using the optogenetic stimulation system (FIG. 12).

### Spike Sorting

To reveal the correlation between light stimulation and neuronal response, data was first bandpass filtered with 300-6,000 Hz, then spike sorted by Kilosort, which was then curated in Phy (available at https://github.com/cortex-lab/phy) to accept, remove, split, or merge units based on their features like waveform template and interspike interval (ISI) distribution. Noise-like units were removed from the results to avoid false positive analysis.

Raster Plot with Light Stimulation

FIG. 7 shows the raster plot for all the firing units with their aggregated firing rate. Optogenetic stimulation was aligned on the plot using blue color with a level of transparency indicating the light intensity. Color-coded units matched the ones on the functional connectivity map.

### Footprints

The HD-MEA provides multiple electrodes that can pick up a signal from the same firing unit because of the narrow spacing. This feature was used to plot the footprints for the color-coded units in FIG. 8. Each dot on the functional connectivity map denotes a spiking unit found from that electrode. Meanwhile, the neighboring electrodes also recorded its activity. The waveform shapes on the footprint indicate that the selected units are from spiking neurons.

### Functional Connectivity Map

FIG. 8 also shows the functional connectivity map with footprints from selected units. To find the correlation between firing units, the spike time tiling coefficient (STTC) was calculated between all pairs with a time window of 20 ms. The STTC ranges from 0 to 1; a more significant number denoted a higher correlation. In the analysis, a threshold was set to 0.35 and defined the units with STTC passing this threshold as functionally connected. Four units were selected from a functionally connected network and their footprints were illustrated along with their firing activity on the raster.

It will be appreciated that of the above-disclosed and other features and functions, or alternatives thereof, may be desirably combined into many other different systems or applications. Also, various presently unforeseen or unanticipated alternatives, modifications, variations, or improvements therein may be subsequently made by those skilled in the art which are also intended to be encompassed by the following claims. Unless specifically recited in a claim, steps, or components according to claims should not be implied or imported from the specification or any other claims as to any particular order, number, position, size, shape, angle, or material.

What is claimed is:

1. An optogenetic stimulation and electrophysiological recording system comprising:
an electrophysiological device coupled to optically active neural tissue, the electrophysiological device having a microelectrode array (MEA) configured to measure electrophysiological signals;
an optical stimulation device configured to emit light configured to stimulate the neural tissue; and
a computing device coupled to the electrophysiological device and the optical stimulation device, the computing device configured to:
control the optical stimulation device to emit the light to stimulate the neural tissue; and
simultaneously record electrophysiological signals from the optically stimulated neural tissue through the electrophysiological device.

2. The system according to claim 1, wherein the neural tissue is an organoid expressing an opsin activatable by the light at a specific wavelength.

3. The system according to claim 2, wherein the opsin is at least one of halorhodopsin, archaerhodopsin, leptosphaeria rhodopsin, a channelrhodopsin, or derivatives thereof.

4. The system according to claim 1, wherein the electrophysiological device includes a well and the MEA is disposed within the well, with the neural tissue disposed on the MEA.

5. The system according to claim 4, wherein the optical stimulation device includes:
a light emitting diode (LED) configured to emit the light;
a fiber optic cable coupled to the LED; and
a well insert assembly insertable into the well and configured to position an end of the fiber optic cable to emit the light in a spot over the neural tissue disposed on the MEA.

6. The system according to claim 5, wherein the LED is configured to emit the light having a wavelength from about 385 nm to about 625 nm.

7. The system according to claim 4, wherein the computing device further includes a display screen configured to display a graphical user interface (GUI).

8. The system according to claim 7, wherein the computing device is further configured to:
generate a grid corresponding to the MEA based on the received electrophysiological signals, the grid including a plurality of footprint units.

9. The system according to claim 8, wherein the grid is displayed on the GUI.

10. The system according to claim 9, wherein each footprint unit of the plurality of footprint units includes a waveform identifier selected from a plurality of waveform identifiers.

11. The system according to claim 8, wherein the computing device is further configured to:
generate a functional connectivity map between a plurality of neural units of the neural tissue.

12. The system according to claim 11, wherein the functional connectivity map is displayed on the GUI.

13. A method for optogenetic stimulation and electrophysiological recording, the method comprising:
emitting light through an optical stimulation device to optically stimulate neural tissue disposed on a microelectrode array (MEA) of an electrophysiological device; and
while emitting the light, recording electrophysiological signals from the optically stimulated neural tissue through the electrophysiological device at a computing device, wherein the computing device is configured to control the optical stimulation device and the electrophysiological device.

14. The method according to claim 13, further comprising:
spike sorting the electrophysiological signals.

15. The method according to claim 13, further comprising:
generating a grid corresponding to the MEA based on the received electrophysiological signals, the grid including a plurality of footprint units.

16. The method according to claim 15, further comprising:
displaying the grid on a graphical user interface (GUI) that is output on a display of the computing device.

17. The method according to claim 15, further comprising:
generating a functional connectivity map between a plurality of neural units of the neural tissue.

18. The method according to claim 17, further comprising:
displaying the functional connectivity map on the GUI that is output on the display of the computing device.
